(19) United States
(12) Patent Application Publication
(10) Pub. No.: US 2026/0096764 A1
(43) Pub. Date: Apr. 9, 2026

(54) BABY ELECTRODE PATCH

(71) Applicant: QT Medical, Inc., Taipei City (TW)

(72) Inventors: Yu-Ting CHEN, Taipei City (TW); Ying-Lung CHENG, Taipei City (TW)

(21) Appl. No.: 19/312,340

(22) Filed: Aug. 28, 2025

(30) Foreign Application Priority Data
Oct. 9, 2024 (TW) 113138559

(51) Int. Cl. A61B 5/282; A61B 5/00; A61B 5/257; A61B 5/268

(52) U.S. Cl. CPC A61B 5/282; A61B 5/257; A61B 5/268; A61B 5/6833; A61B 2503/04; A61B 2560/0468; A61B 2562/227

(57) ABSTRACT

A baby electrode patch is connected to an electrocardiogram measurement equipment to measure an electrocardiogram of a baby. The baby electrode patch included an electrode patch main body, an electrode patch extension portion, and an electrode patch connection portion. The electrode patch main body is equipped with a plurality of main body electrodes. The electrode patch extension portion is connected to the electrode patch main body, and the electrode patch connection portion is connected to the electrode patch extension portion. The electrode patch extension portion extends from the electrode patch main body towards a side of the baby so as to allow the electrocardiogram measurement equipment to be placed at the side of the baby.

### RELATED APPLICATIONS

This application claims priority to Taiwan Application Serial Number 113138559 filed October 09, 2024, the disclosures of which are incorporated herein by reference in their entireties.

### BACKGROUND

### Technical Field

The present invention relates to an electrode patch. More particularly, the present invention relates to a baby electrode patch.

### Description of Related Art

With the increasing advancement of technology and medical techniques, medical equipment has also advanced, providing precise measurements and diagnoses not only for adults and the elderly. Advanced medical equipment may also provide more accurate measurements for children and babies.

Arrhythmia is a common symptom in heart-related diseases, making the monitoring of heart-related diseases an important issue for humans. Currently, the detection of heart rate/electrocardiogram primarily uses electrocardiogram measurement equipment for monitoring. The traditional method involves attaching wet electrodes containing electrolytes to the chest to serve as a connection between the skin and the machine, thereby measuring heart rate/electrocardiogram.

However, with the advancement of technology, the electrocardiogram measurement equipment has been gradually developed. Disposable electrode patches may quickly and conveniently perform electrocardiogram measurements. In addition, the body proportion of the baby is completely different from that of the adult, even with smaller electrocardiogram patches, measurement data errors still occur due to the instability of babies during measurement.

Therefore, there is a need to provide an electrocardiogram electrode patch suitable for babies.

### SUMMARY

One object of the present invention is to provide a baby electrode patch able to prevent the electrocardiogram (ECG) measurement equipment from pressing on the body of the baby so as to avoid discomfort for the baby and therefore effectively improve the accuracy and convenience of baby electrocardiogram measurement.

To achieve these and other advantages and in accordance with the objective of the embodiments of the present invention, as the embodiment broadly describes herein, the embodiments of the present invention provides a baby electrode patch for connecting to an electrocardiogram measurement equipment to measure an electrocardiogram of the baby. The baby electrode patch includes an electrode patch main body, an electrode patch extension portion, and an electrode patch connection portion. The electrode patch main body is equipped with a plurality of main body electrodes. The electrode patch extension portion is connected to the electrode patch main body, and the electrode patch connection portion is connected to the electrode patch extension portion. The electrode patch extension portion is configured to extend from the electrode patch main body towards a side of the baby so as to allow the electrocardiogram measurement equipment to be placed at the side of the baby.

In some embodiments, the baby electrode patch for connecting to an electrocardiogram (ECG) measurement equipment to measure an electrocardiogram of a baby includes an electrode patch main body, an electrode patch extension portion, and an electrode patch connection portion. The electrode patch main body is equipped with a plurality of main body electrodes. The electrode patch extension portion is connected to the electrode patch main body. The electrode patch connection portion is connected to the electrode patch extension portion. The electrode patch extension portion is configured to extend horizontally from the electrode patch main body towards a side of the baby, so that the electrocardiogram measurement equipment is placed at the side of the baby.

In some embodiments, the baby electrode patch further includes a plurality of elliptical hydrogel layers, respectively connected to corresponding main body electrodes of the main body electrodes.

In some embodiments, each of the elliptical hydrogel layers includes a major axis and a minor axis, and the major axes of the elliptical hydrogel layers are parallel to each other.

In some embodiments, the baby electrode patch further includes a plurality of conductive traces, respectively connected to corresponding main body electrodes of the main body electrodes and extending to the electrode patch connection portion.

In some embodiments, the baby electrode patch further includes a plurality of wear-resistant conductive layers located at the electrode patch connection portion and respectively covering corresponding conductive traces of the conductive traces.

In some embodiments, the baby electrode patch further includes a first insulating layer, and the conductive traces and the main body electrodes are formed on the first insulating layer.

In some embodiments, the baby electrode patch further includes a second insulating layer, covering the conductive traces and the first insulating layer.

In some embodiments, the baby electrode patch further includes a skin-friendly contact layer formed on the second insulating layer.

In some embodiments, the second insulating layer and the skin-friendly contact layer include at least one opening.

In some embodiments, the at least one opening includes a plurality of openings, respectively exposing the main body electrodes.

In some embodiments, the at least one opening is an integrated opening to simultaneously expose the main body electrodes.

In some embodiments, the baby electrode patch further includes a plurality of extension conductive traces, a plurality of extension electrodes and a plurality of elliptical hydrogel layers. The extension conductive traces are extending from the electrode patch connection portion, passing through the electrode patch main body and then extending outward. The extension electrodes are respectively connected to corresponding extension conductive traces of the extension conductive traces. The elliptical hydrogel layers are respectively connected to corresponding extension electrodes of the extension electrodes.

In some embodiments, portions of the conductive traces and the first insulating layer located at the electrode patch connection portion are folded and fixed to a back side of the first insulating layer.

In some embodiments, the second insulating layer covers the portions of the conductive traces located at the electrode patch connection portion on a front side of the first insulating layer, and exposes other portions of the conductive traces located at the electrode patch connection portion on the front side of the first insulating layer.

In some embodiments, the wear-resistant conductive layers are respectively formed on surfaces of the portions of the conductive traces located on the back side of the first insulating layer, and on surfaces of the other portions of the conductive traces located at the electrode patch connection portion on the front side of the first insulating layer.

According to another aspect of the present invention, a baby electrode patch for connecting to an electrocardiogram (ECG) measurement equipment to measure an electrocardiogram of a baby includes an electrode patch main body, an electrode patch extension portion, an electrode patch connection portion and a plurality of elliptical hydrogel layers. The electrode patch main body is equipped with a plurality of main body electrodes. The electrode patch extension portion is connected to the electrode patch main body. The electrode patch connection portion is connected to the electrode patch extension portion, and the electrode patch extension portion is configured to extend horizontally from the electrode patch main body towards a side of the baby, so that the electrocardiogram measurement equipment is placed at the side of the baby. The elliptical hydrogel layers are respectively connected to corresponding main body electrodes of the main body electrodes. In addition, each of the elliptical hydrogel layers includes a major axis and a minor axis, and the major axes of the elliptical hydrogel layers are parallel to each other.

In some embodiments, the baby electrode patch further includes a plurality of conductive traces, a plurality of wear-resistant conductive layers, a first insulating layer, a second insulating layer and a skin-friendly contact layer. The conductive traces are respectively connected to corresponding main body electrodes of the main body electrodes and extending to the electrode patch connection portion. The wear-resistant conductive layers are located at the electrode patch connection portion and respectively covering corresponding conductive traces of the conductive traces. The conductive traces and the main body electrodes are formed on the first insulating layer. The second insulating layer covers the conductive traces and the first insulating layer and the skin-friendly contact layer is formed on the second insulating layer. The second insulating layer and the skin-friendly contact layer include at least one opening, the at least one opening includes a plurality of openings, respectively exposing the main body electrodes, and the at least one opening is an integrated opening to simultaneously expose the main body electrodes.

In some embodiments, the baby electrode patch further includes a plurality of extension conductive traces, a plurality of extension electrodes and a plurality of elliptical hydrogel layers. The extension conductive traces are extending from the electrode patch connection portion, passing through the electrode patch main body and then extending outward. The extension electrodes are respectively connected to corresponding extension conductive traces of the extension conductive traces and the elliptical hydrogel layers are respectively connected to corresponding extension electrodes of the extension electrodes.

In some embodiments, portions of the conductive traces and the first insulating layer located at the electrode patch connection portion are folded and fixed to a back side of the first insulating layer.

In some embodiments, the second insulating layer covers the portions of the conductive traces located at the electrode patch connection portion on a front side of the first insulating layer, and exposes other portions of the conductive traces located at the electrode patch connection portion on the front side of the first insulating layer, and the wear-resistant conductive layers are respectively formed on surfaces of the portions of the conductive traces located on the back side of the first insulating layer, and on surfaces of the other portions of the conductive traces located at the electrode patch connection portion on the front side of the first insulating layer.

Hence, the baby electrode patch as mentioned above may be easily attached to the skin surface of the chest of a baby so as to improve the accuracy and stability of electrode patch measurements. Furthermore, by utilizing the electrode patch extension portion, the electrocardiogram measurement equipment may place outside the body of the baby so that the discomfort for the baby during electrocardiogram measurement may be further reduced.

### BRIEF DESCRIPTION OF THE DRAWINGS

The disclosure may be more fully understood by reading the following detailed description of the embodiment, with reference made to the accompanying drawings as follows:

FIG. 1 is a schematic diagram illustrating a baby electrode patch attached to a body of a baby according to an embodiment of the present invention;

FIG. 2 is a schematic front view of a baby electrode patch according to an embodiment of the present invention;

FIG. 3 is a schematic back view of a baby electrode patch according to an embodiment of the present invention;

FIG. 4 is a schematic back view of a baby electrode patch according to another embodiment of the present invention; and

FIG. 5 is a partial schematic front and back view of a folded connection terminal of a baby electrode patch according to yet another embodiment of the present invention.

### DETAILED DESCRIPTION OF THE PREFERRED EMBODIMENT

The following is a detailed description of the embodiments in conjunction with the accompanying drawings, but the provided embodiments are not intended to limit the scope of the disclosure, and the description of the structure and operation is not utilized to limit the execution sequence thereof. The structure of the recombination of components and the resulting devices with equal functions are all within the scope of this disclosure. In addition, the drawings are for illustration purposes only, and are not drawn according to the original scale. For ease of understanding, the same reference numbers are utilized in the drawings and the description to refer to the same or like parts.

In addition, the terms utilized in the entire description and the scope of the patent application, unless otherwise specified, usually have the usual meaning of each term utilized in this field, in the content disclosed here and in the special content. Some terms utilized to describe the disclosure are discussed below or elsewhere in this specification to provide additional guidance to those skilled in the art in the disclosure.

In the implementation mode and the scope of the present application, unless the article is specifically limited in the context, "a" and "the" may generally refer to a single or pluralities. In the steps, the numbering is only utilized to conveniently describe the steps, rather than to limit the sequence and implementation.

Secondly, the words "comprising", "including", "having", "containing" and the like utilized in the present application are all open language, meaning including but not limited thereto.

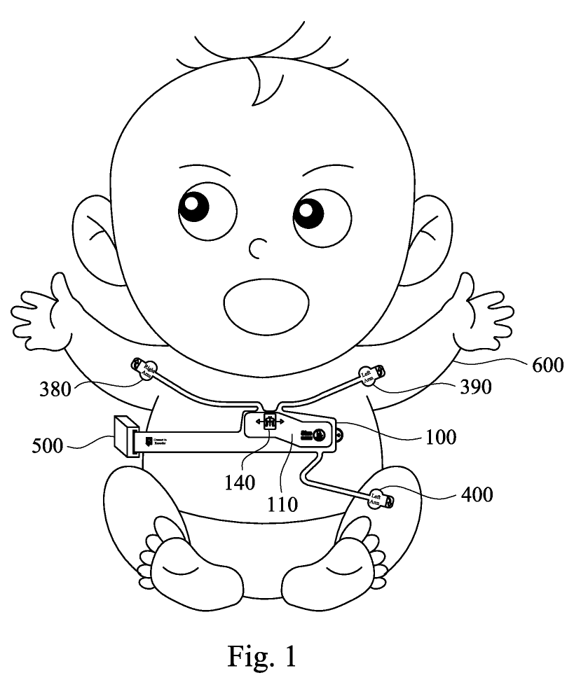

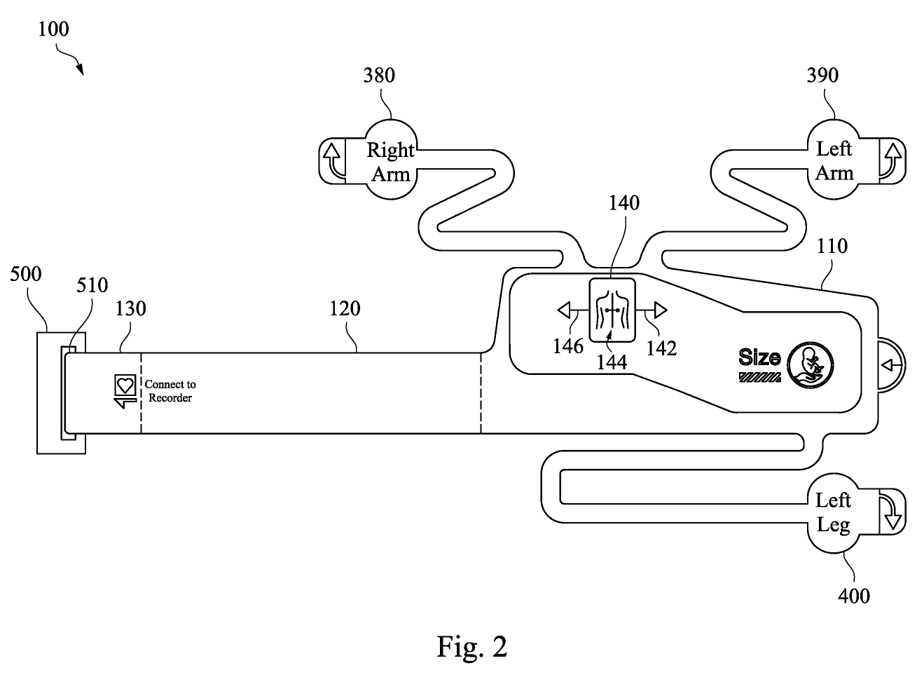

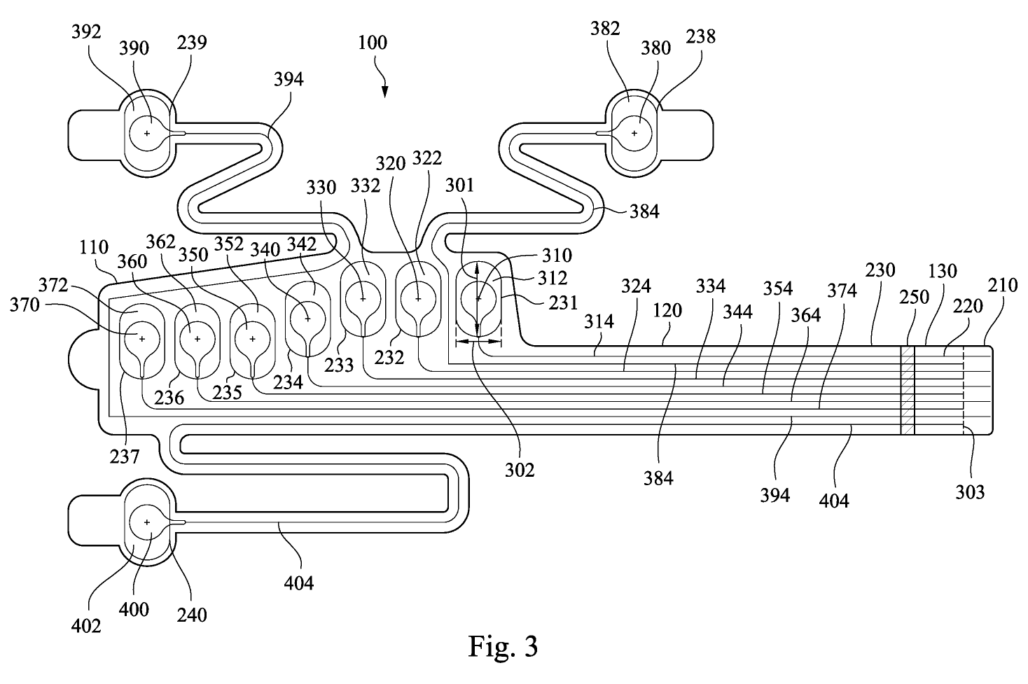

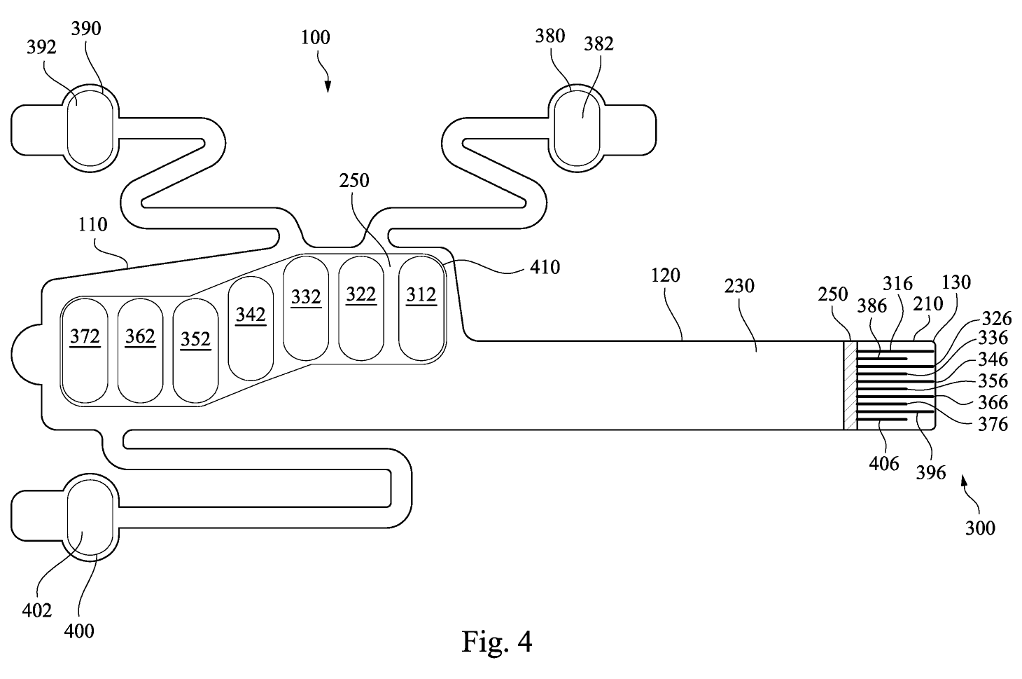

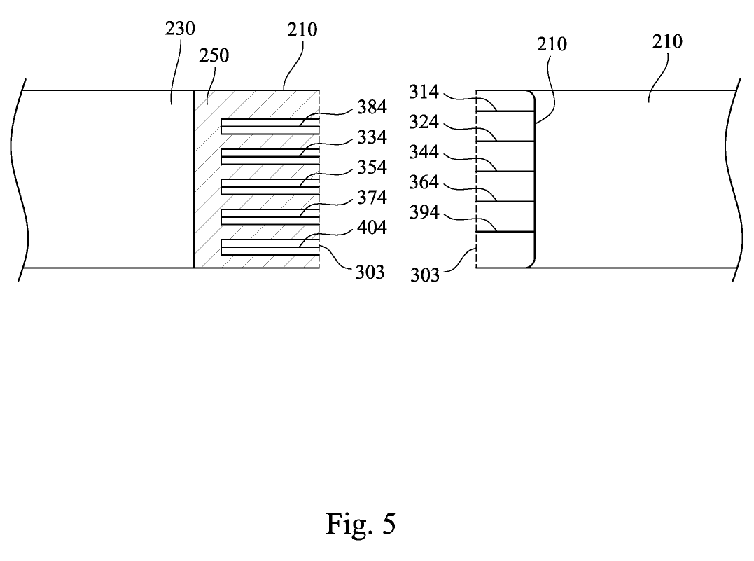

FIG. 1 is a schematic diagram illustrating a baby electrode patch attached to a body of a baby according to an embodiment of the present invention, FIG. 2 is a schematic front view thereof, and FIG. 3 is a schematic back view thereof. In addition, FIG. 4 is a schematic back view of a baby electrode patch according to another embodiment of the present invention. Furthermore, FIG. 5 is a partial schematic front and back view of a folded connection terminal of a baby electrode patch according to yet another embodiment of the present invention.

Referring to FIGS. 1 and 2, a schematic diagram shows the baby electrode patch 100 attached to the body of a baby 600. First, the central position mark 144 of the positioning indicator 140 on the electrode patch main body 110 of the baby electrode patch 100 is aligned with the center of the chest of the baby 600, and the first arrow 142 and the second arrow 146 of the positioning indicator 140 are respectively aligned with the positions of the left and right nipples of the baby 600.

In addition, the baby electrode patch 100 is further connected to the electrocardiogram measurement equipment 500 to measure the electrocardiogram of a baby 600.

It is worth noting that, at this time, the electrocardiogram (ECG) measurement equipment 500 may therefore be placed at a side of the baby 600, instead of pressing on the body of the baby 600 so as to effectively avoid discomfort caused by the pressure of the electrocardiogram measurement equipment 500 pressing on the baby 600, which might affect the accuracy and stability of the electrocardiogram measurement.

The baby electrode patch 100 includes an electrode patch main body 110, an electrode patch extension portion 120, and an electrode patch connection portion 130. The electrode patch main body 110 is equipped with a plurality of main body electrodes. The electrode patch extension portion 120 is connected to the electrode patch main body 110, and the electrode patch connection portion 130 is connected to the electrode patch extension portion 120.

Referring simultaneously to FIG. 3, as shown in the figure, the main body electrodes on the electrode patch main body 110 include, for example, a first main body electrode 310, a second main body electrode 320, a third main body electrode 330, a fourth main body electrode 340, a fifth main body electrode 350, a sixth main body electrode 360, and a seventh main body electrode 370.

In some embodiments, the first main body electrode 310 represents the RL electrode, the second main body electrode 320 represents the V1 electrode, the third main body electrode 330 represents the V2 electrode, the fourth main body electrode 340 represents the V3 electrode, the fifth main body electrode 350 represents the V4 electrode, the sixth main body electrode 360 represents the V5 electrode, and the seventh main body electrode 370 represents the V6 electrode.

Therefore, the electrode patch extension portion 120 of the baby electrode patch 100 extends from the electrode patch main body 110 towards one side of the baby 600, for example, horizontally extends from the electrode patch main body 110 towards the side of the baby 600, so that the electrocardiogram measurement equipment 500 may be placed at the side of the baby 600, rather than on the body of the baby 600.

In addition, the baby electrode patch 100 further includes a plurality of extension conductive traces, e.g. a first extension conductive trace 384, a second extension conductive trace 394, and a third extension conductive trace 404, as well as a plurality of extension electrodes, such as a first extension electrode 380, a second extension electrode 390, and a third extension electrode 400. In some embodiments, the first extension conductive trace 384 extends from the electrode patch connection portion 130, passes through the electrode patch main body 110 and then extends outward. In addition, the first extension electrode 380 is connected to the first extension conductive trace 384, to measure the electrocardiogram signal on the right hand of the baby 600. Similarly, the second extension electrode 390 is used to measure the electrocardiogram signal on the left hand of the baby 600, and the third extension electrode 400 may measure the electrocardiogram signal of the left leg of the baby 600.

In some embodiments, the first extension electrode 380 is an RA extension electrode, the second extension electrode 390 is an LA extension electrode, and the third extension electrode 400 is an LL extension electrode.

In some embodiments, the baby electrode patch 100 further includes a plurality of elliptical hydrogel layers respectively connected to corresponding main body electrodes and extension electrodes. For example, the first elliptical hydrogel layer 312 covers the first main body electrode 310 and is electrically connected to the first main body electrode 310. The second elliptical hydrogel layer 322 covers the second main body electrode 320 and is electrically connected to the second main body electrode 320. The third elliptical hydrogel layer 332 covers the third main body electrode 330 and is electrically connected to the third main body electrode 330. The fourth elliptical hydrogel layer 342 covers the fourth main body electrode 340 and is electrically connected to the fourth main body electrode 340. The fifth elliptical hydrogel layer 352 covers the fifth main body electrode 350 and is electrically connected to the fifth main body electrode 350. The sixth elliptical hydrogel layer 362 covers the sixth main body electrode 360 and is electrically connected to the sixth main body electrode 360. The seventh elliptical hydrogel layer 372 covers the seventh main body electrode 370 and is electrically connected to the seventh main body electrode 370.

Furthermore, the first extension elliptical hydrogel layer 382 covers the first extension electrode 380 and is electrically connected to the first extension electrode 380. The second extension elliptical hydrogel layer 392 covers the second extension electrode 390 and is electrically connected to the second extension electrode 390. The third extension elliptical hydrogel layer 402 covers the third extension electrode 400 and is electrically connected to the third extension electrode 400.

In some embodiments, taking the first elliptical hydrogel layer 312 and the first main body electrode 310 as an example, the first elliptical hydrogel layer 312 includes a major axis 301 and a minor axis 302. It is worth noting that, in particular, the major axes 301 of the elliptical hydrogel layers of the plurality of main body electrodes on the electrode patch main body 110 are parallel to each other. Preferably, the major axes 301 of the elliptical hydrogel layers of the main body electrodes are aligned with the height direction of the baby 600.

In some embodiments, the major axes 301 of the first extension elliptical hydrogel layer 382, the second extension elliptical hydrogel layer 392, and the third extension elliptical hydrogel layer 402 are also parallel to each other and aligned with the height direction of the baby 600, but the present invention is not limited thereto.

Therefore, the elliptical hydrogel layer may effectively increase the area attached to the body of the baby 600 and effectively transmit voltage signals. In addition, by reducing the length of the elliptical hydrogel layer in the minor axis 302 direction, the baby electrode patch 100 may effectively reduce the spacing between the main body electrodes, allowing the main body electrodes to be placed closer together, thereby enabling the baby electrode patch 100 to be attached to the chest of the baby 600 so as to effectively improve the accuracy and convenience of the electrocardiogram measurement.

In some embodiments, the baby electrode patch 100 further includes a metal conductive trace layer 220, including a plurality of conductive traces. The conductive traces are respectively connected to corresponding main body electrodes and extension electrodes, and extend to the electrode patch connection portion 130. In addition, a wear-resistant conductive layer 300 is preferably disposed on the electrode patch connection portion 130 and covers the corresponding conductive traces to increase the durability of the baby electrode patch 100.

Therefore, the baby electrode patch 100 may utilize the wear-resistant conductive layer 300 to electrically connect to the connector 510 of the electrocardiogram measurement equipment 500 when the electrode patch connection portion 130 is inserted into the connector 510 of the electrocardiogram measurement equipment 500. The wear-resistant conductive layer 300 increases the wear resistance and reliability of the baby electrode patch 100, thereby improving the electrical and mechanical properties of the baby electrode patch 100.

For example, the first conductive trace 314 is connected to the first main body electrode 310 and extends to the electrode patch connection portion 130, and the first wear-resistant conductive portion 316 is formed on the first conductive trace 314 on the electrode patch connection portion 130. The first wear-resistant conductive portion 316 effectively protects the first conductive trace 314 on the electrode patch connection portion 130, and the first conductive trace 314 enhances the conductivity of the conductive trace, thereby improving the accuracy and stability of the measurement of the baby electrode patch 100.

In some embodiments, the first conductive trace 314 may be a nano-silver paste circuit layer or a copper metal layer. In addition, the first wear-resistant conductive portion 316 may be a conductive graphene layer, without departing from the spirit and scope of protection of the present invention.

Similarly, the second wear-resistant conductive portion 326 is formed on the second conductive trace 324 on the electrode patch connection portion 130. The third wear-resistant conductive portion 336 is formed on the third conductive trace 334 on the electrode patch connection portion 130. The fourth wear-resistant conductive portion 346 is formed on the fourth conductive trace 344 on the electrode patch connection portion 130. The fifth wear-resistant conductive portion 356 is formed on the fifth conductive trace 354 on the electrode patch connection portion 130. The sixth wear-resistant conductive portion 366 is formed on the sixth conductive trace 364 on the electrode patch connection portion 130. The seventh wear-resistant conductive portion 376 is formed on the seventh conductive trace 374 on the electrode patch connection portion 130.

In addition, the first extension wear-resistant conductive portion 386 is formed on the first extension conductive trace 384 on the electrode patch connection portion 130. The second extension wear-resistant conductive portion 396 is formed on the second extension conductive trace 394 on the electrode patch connection portion 130. The third extension wear-resistant conductive portion 406 is formed on the third extension conductive trace 404 on the electrode patch connection portion 130.

In some embodiments, simultaneously referring to FIGS. 1 to 3, the baby electrode patch 100 further includes a first insulating layer 210. The conductive traces, the main body electrodes and the extension electrodes are preferably all formed on the first insulating layer 210.

Furthermore, in some embodiments, the baby electrode patch 100 further includes a second insulating layer 250, covering the conductive traces, the main body electrodes, the extension electrodes and the first insulating layer 210, and the second insulating layer 250 exposes the conductive traces and the wear-resistant conductive portions on the electrode patch connection portion 130.

In some embodiments, the first conductive trace 314, the first wear-resistant conductive portion 316, the second conductive trace 324, the second wear-resistant conductive portion 326, the third conductive trace 334, the third wear-resistant conductive portion 336, the fourth conductive trace 344, the fourth wear-resistant conductive portion 346, the fifth conductive trace 354, the fifth wear-resistant conductive portion 356, the sixth conductive trace 364, the sixth wear-resistant conductive portion 366, the seventh conductive trace 374, the seventh wear-resistant conductive portion 376, the first extension conductive trace 384, the first extension wear-resistant conductive portion 386, the second extension conductive trace 394, the second extension wear-resistant conductive portion 396, the third extension conductive trace 404 and the third extension wear-resistant conductive portion 406 are preferably formed on one surface of the first insulating layer 210, or may be formed on the front and back sides of the first insulating layer 210, without departing from the spirit and scope of protection of the present invention.

In some embodiments, simultaneously referring to FIGS. 3 and 5, the first conductive trace 314, the second conductive trace 324, the third conductive trace 334, the fourth conductive trace 344, the fifth conductive trace 354, the sixth conductive trace 364, the seventh conductive trace 374, the first extension conductive trace 384, the second extension conductive trace 394, and the third extension conductive trace 404 may be formed on one surface of the first insulating layer 210, and the first conductive trace 314, the second conductive trace 324, the fourth conductive trace 344, the sixth conductive trace 364, the second extension conductive trace 394, and the first insulating layer 210, extending beyond the end faces of the third conductive trace 334, the fifth conductive trace 354, the seventh conductive trace 374, the first extension conductive trace 384 and the third extension conductive trace 404, may be folded along the folding line 303 and fixed to the back side of the first insulating layer 210 (refer to the diagram on the right side of FIG. 5). The second insulating layer 250 further extends and covers portions of the first conductive trace 314, the second conductive trace 324, the fourth conductive trace 344, the sixth conductive trace 364, and the second extension conductive trace 394 located on the front side of the first insulating layer 210, thereby only exposing the first extension conductive trace 384, the third conductive trace 334, the fifth conductive trace 354, the seventh conductive trace 374, and the third extension conductive trace 404 (referring to the diagram on the left side of FIG. 5). Therefore, the connection terminals formed by the conductive traces have a larger spacing and be evenly distributed on the front and back sides of the first insulating layer 210, thereby improving the insulation performance between the conductive traces and further improving the signal quality and measurement accuracy of the baby electrode patch 100. The folding line 303 is drawn with a dashed line, which only indicates the folding position and does not need to be present on the surface of the baby electrode patch 100, but not limited thereto.

In some embodiments, the first wear-resistant conductive portion 316, the second wear-resistant conductive portion 326, the third wear-resistant conductive portion 336, the fourth wear-resistant conductive portion 346, the fifth wear-resistant conductive portion 356, the sixth wear-resistant conductive portion 366, the seventh wear-resistant conductive portion 376, the first extension wear-resistant conductive portion 386, the second extension wear-resistant conductive portion 396, and the third extension wear-resistant conductive portion 406 may be formed on the surface of the corresponding conductive traces exposed from the second insulating layer 250 to increase conductivity and wear resistance thereof.

In some embodiments, the first insulating layer 210 and the second insulating layer 250 may be formed of an insulating material such as a polyethylene terephthalate (PET) plastic film, a polyurethane (PU) plastic film, a polyvinyl chloride (PVC) plastic film, a polycarbonate (PC) plastic film, or a hard paperboard.

In some embodiments, the second insulating layer 250 may be an insulating paint coated on the surfaces of the conductive traces and the first insulating layer 210, but not limited thereto.

In some embodiments, the baby electrode patch 100 further includes a skin-friendly contact layer 230, formed on the second insulating layer 250. In some embodiments, the skin-friendly contact layer 230 may include a non-woven fabric contact layer. In some embodiments, the second insulating layer 250 may be disposed on the inner side of the skin-friendly contact layer 230 and attached to the conductive traces and the first insulating layer 210.

In some embodiments, the second insulating layer 250 and the skin-friendly contact layer 230 include at least one opening.

Referring to FIG. 3, as shown in the figure, the at least one opening may include a first opening 231, a second opening 232, a third opening 233, a fourth opening 234, a fifth opening 235, a sixth opening 236, a seventh opening 237, a first extension opening 238, a second extension opening 239, and a third extension opening 240, to respectively expose the underlying main body electrodes and extension electrodes.

In addition, referring to FIG. 4, as shown in the figure, the openings on the electrode patch main body 110 of the baby electrode patch 100 may use a main body integrated opening 410 instead of the first opening 231, the second opening 232, the third opening 233, the fourth opening 234, the fifth opening 235, the sixth opening 236 and the seventh opening 237, or some of the aforementioned openings shown in FIG. 3, to facilitate the manufacturing of the baby electrode patch 100, without departing from the spirit and scope of protection of the present invention.

In some embodiments, the first insulating layer 210, the metal conductive trace layer 220, the skin-friendly contact layer 230, and the second insulating layer 250 are respectively formed by a same piece of insulating material, metal material, and skin-friendly material, but not limited thereto.

In some embodiments, the electrode patch main body 110 may be adhered to the body of the baby being examined by a self-adhesive backing.

It is worth noting that the electrodes formed on the electrode patch main body disclosed in the present invention, further including the three extension electrodes, may therefore accurately measure the an electrocardiogram of the baby. Hence, the medical personnel may easily attach the baby electrode patch of the present invention to the body of the baby and correctly perform electrocardiogram measurements.

According to the baby electrode patch disclosed in the present invention, the electrode patch may be conveniently attached to the surface of the chest of the baby so as to improve the accuracy and stability of electrode patch measurements. In addition, by utilizing the electrode patch extension portion to place the electrocardiogram measurement equipment outside the body of the baby, discomfort for the baby during electrocardiogram measurement may be further reduced.

Although the present disclosure has been disclosed above in terms of implementation, it is not intended to limit the present disclosure. Any person with ordinary knowledge in the field may make various variations and modifications without departing from the spirit and scope of the disclosure. Therefore, the spirit and scope of the appended claims should not be limited to the description of the embodiments contained herein.

What is claimed is:

1. A baby electrode patch for connecting to an electrocardiogram (ECG) measurement equipment to measure an electrocardiogram of a baby, comprising:
an electrode patch main body equipped with a plurality of main body electrodes;
an electrode patch extension portion connected to the electrode patch main body; and
an electrode patch connection portion connected to the electrode patch extension portion, wherein the electrode patch extension portion is configured to extend horizontally from the electrode patch main body towards a side of the baby, so that the electrocardiogram measurement equipment is placed at the side of the baby.

2. The baby electrode patch of claim 1, further comprising:
a plurality of elliptical hydrogel layers, respectively connected to corresponding main body electrodes of the main body electrodes.

3. The baby electrode patch of claim 2, wherein each of the elliptical hydrogel layers comprises:
a major axis; and
a minor axis, wherein the major axes of the elliptical hydrogel layers are parallel to each other.

4. The baby electrode patch of claim 2, further comprising:
a plurality of conductive traces, respectively connected to corresponding main body electrodes of the main body electrodes and extending to the electrode patch connection portion.

5. The baby electrode patch of claim 4, further comprising:
a plurality of wear-resistant conductive layers located at the electrode patch connection portion and respectively covering corresponding conductive traces of the conductive traces.

6. The baby electrode patch of claim 5, further comprising:
a first insulating layer, wherein the conductive traces and the main body electrodes are formed on the first insulating layer.

7. The baby electrode patch of claim 6, further comprising:
a second insulating layer, covering the conductive traces and the first insulating layer.

8. The baby electrode patch of claim 7, further comprising:
a skin-friendly contact layer formed on the second insulating layer.

9. The baby electrode patch of claim 8, wherein the second insulating layer and the skin-friendly contact layer comprise at least one opening.

10. The baby electrode patch of claim 9, wherein the at least one opening comprises a plurality of openings, respectively exposing the main body electrodes.

11. The baby electrode patch of claim 10, wherein the at least one opening is an integrated opening to simultaneously expose the main body electrodes.

12. The baby electrode patch of claim 9, further comprising:
a plurality of extension conductive traces, extending from the electrode patch connection portion, passing through the electrode patch main body and then extending outward;
a plurality of extension electrodes, respectively connected to corresponding extension conductive traces of the extension conductive traces; and
a plurality of elliptical hydrogel layers, respectively connected to corresponding extension electrodes of the extension electrodes.

13. The baby electrode patch of claim 7, wherein portions of the conductive traces and the first insulating layer located at the electrode patch connection portion are folded and fixed to a back side of the first insulating layer.

14. The baby electrode patch of claim 13, wherein the second insulating layer covers the portions of the conductive traces located at the electrode patch connection portion on a front side of the first insulating layer, and exposes other portions of the conductive traces located at the electrode patch connection portion on the front side of the first insulating layer.

15. The baby electrode patch of claim 14, wherein the wear-resistant conductive layers are respectively formed on surfaces of the portions of the conductive traces located on the back side of the first insulating layer, and on surfaces of the other portions of the conductive traces located at the electrode patch connection portion on the front side of the first insulating layer.

16. A baby electrode patch for connecting to an electrocardiogram (ECG) measurement equipment to measure an electrocardiogram of a baby, comprising:
an electrode patch main body equipped with a plurality of main body electrodes;
an electrode patch extension portion connected to the electrode patch main body;
an electrode patch connection portion connected to the electrode patch extension portion, wherein the electrode patch extension portion is configured to extend horizontally from the electrode patch main body towards a side of the baby, so that the electrocardiogram measurement equipment is placed at the side of the baby; and
a plurality of elliptical hydrogel layers, respectively connected to corresponding main body electrodes of the main body electrodes, wherein each of the elliptical hydrogel layers comprises:
a major axis; and
a minor axis, wherein the major axes of the elliptical hydrogel layers are parallel to each other.

17. The baby electrode patch of claim 16, further comprising:
a plurality of conductive traces, respectively connected to corresponding main body electrodes of the main body electrodes and extending to the electrode patch connection portion;
a plurality of wear-resistant conductive layers located at the electrode patch connection portion and respectively covering corresponding conductive traces of the conductive traces;
a first insulating layer, wherein the conductive traces and the main body electrodes are formed on the first insulating layer;
a second insulating layer, covering the conductive traces and the first insulating layer; and
a skin-friendly contact layer formed on the second insulating layer, wherein the second insulating layer and the skin-friendly contact layer comprise at least one opening, wherein the at least one opening comprises a plurality of openings, respectively exposing the main body electrodes, wherein the at least one opening is an integrated opening to simultaneously expose the main body electrodes.

18. The baby electrode patch of claim 17, further comprising:
a plurality of extension conductive traces, extending from the electrode patch connection portion, passing through the electrode patch main body and then extending outward;
a plurality of extension electrodes, respectively connected to corresponding extension conductive traces of the extension conductive traces; and
a plurality of elliptical hydrogel layers, respectively connected to corresponding extension electrodes of the extension electrodes.

19. The baby electrode patch of claim 17, wherein portions of the conductive traces and the first insulating layer located at the electrode patch connection portion are folded and fixed to a back side of the first insulating layer.

20. The baby electrode patch of claim 19, wherein the second insulating layer covers the portions of the conductive traces located at the electrode patch connection portion on a front side of the first insulating layer, and exposes other portions of the conductive traces located at the electrode patch connection portion on the front side of the first insulating layer, wherein the wear-resistant conductive layers are respectively formed on surfaces of the portions of the conductive traces located on the back side of the first insulating layer, and on surfaces of the other portions of the conductive traces located at the electrode patch connection portion on the front side of the first insulating layer.
